(19) United States
(12) Patent Application Publication
(10) Pub. No.: US 2020/0221907 A1
(43) Pub. Date: Jul. 16, 2020

(54) Shower Shuffle

(71) Applicant: Keiley Vickers, Virginia Beach, VA (US)

(72) Inventor: Keiley Vickers, Virginia Beach, VA (US)

(21) Appl. No.: 16/244,823

(22) Filed: Jan. 10, 2019

(51) Int. Cl. A47K 3/00; A61B 17/54; A47K 7/02

(52) U.S. Cl. CPC A47K 3/002; A61B 2017/320004; A47K 7/026; A61B 17/54

(57) ABSTRACT

The shower shuffle is a full-size shower mat with enhanced design to include a foot soaking section and a dead skin removal section. The foot soaking section will be ⅓ of the shower mat and made up of a sponge/luffa material. The remaining ⅔ of the Shower Shuffle will be made up of pumice stone for shuffling away dead skin. The bottom will have plastic railings under the sponge section to reinforce safety with weight and level it to the pumice stone. The entire bottom will contain rubber suction cups to keep the full mat in place.

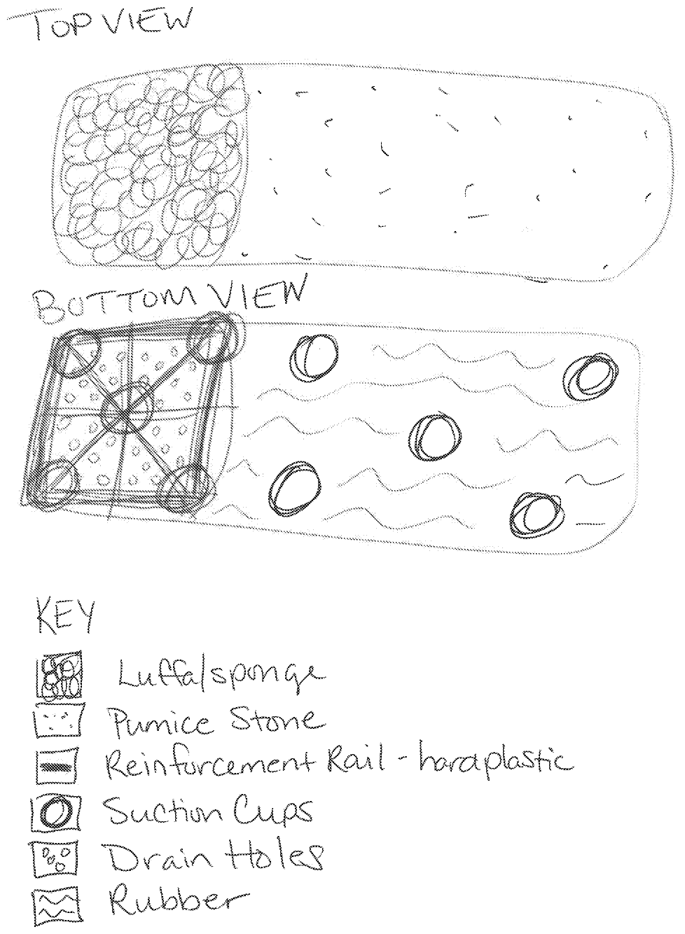

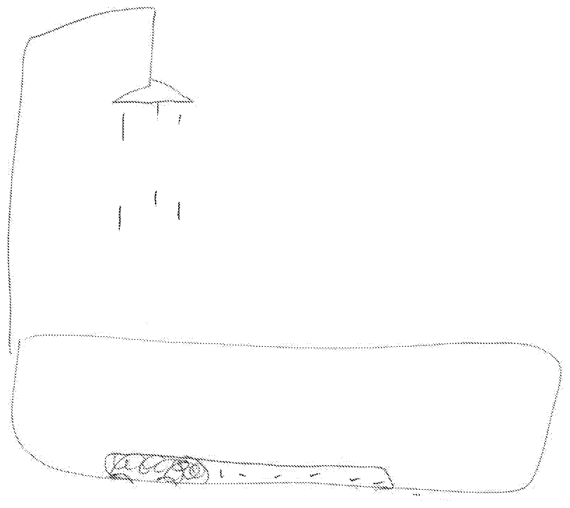

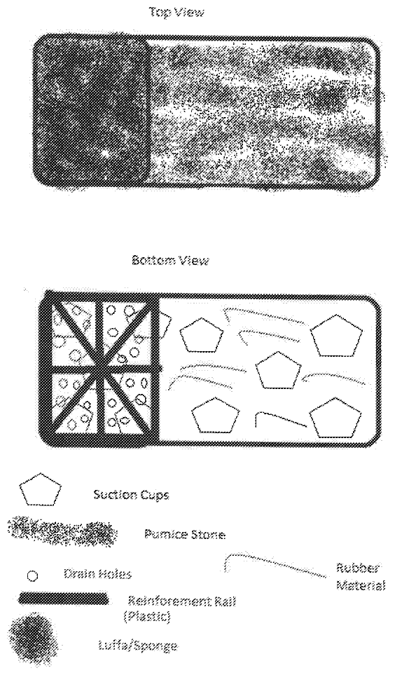

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

This invention pertains to the field of health and beauty. It was thought of to assist all humans with the removal of dead skin and calluses saving them time and effort. It can be used by any BODY. Designed to be used in the shower to prevent bending or spending a fortune for foot maintenance.

Other products known to me were introduced to the market on infomercials after I contacted Davidson Design about my inventions. However, the other inventions are not full mats that a person can stand. Similar inventions contain a single flip for the shower with rubber material for scrubbing and another with a foot file material.

### BRIEF SUMMARY OF THE INVENTION

The Shower Shuffle will be designed to be the size of a shower mat, replacing a standard shower mat. The Shower Shuffle will be designed with rubber suction cups on the bottom to prevent slipping and plastic reinforcement rails under the soaking area. The top of the matt will be designed with majority pumice stone material. A smaller section of the mat will contain soaking sponges to help soften the feet. This will lay in your shower large enough for both feet. As you shower you can soak your feet and then, shuffle your feet against the mat to help with dry, dead skin. This can be of use to people of all ages, size, and health levels while they are showering.

### BRIEF DESCRIPTION OF THE SEVERAL VIEWS OF THE DRAWING

Top of Shower Shuffle horizontal view:

Left side will have a luffa/sponge section that will hold the consumers luffa or bath sponge allowing a consumer to soak their feet during the first part of their shower. This section will be ⅓ the size of the entire mat.

The right side will be all pumice stone, ⅔ of the mat.

Bottom of Shower Shuffle horizontal view:

Left sides where the luffa/sponge compartment sits will be drain holes throughout the bottom along with corner suction cups and plastic reinforcement around and through the center of this section to sustain weight and elevate this section from the shower floor and leveling it with the pumice stone.

Entire right side will be a rubber bottom under the pumice stone with suction cups to hold it in place and prevent the stone from scratching the bathtub floor.

### DETAILED DESCRIPTION OF THE INVENTION

The Shower Shuffle is a shower gadget that will replace a shower mat and have benefits of helping anyone soak and shuffle away dead skin on their feet while showering. At a horizontal view, the top of the matt will have a left side soaking sponges to help soften the feet. This will be about ⅓ of the mat. This sponge can eventually be replaced by the consumer with a luffa or sponge of their choice. The left side, horizontal, bottom view will include drainage holes to allow the replaceable luffa/sponge to properly drain and dry after use. This section will also have a plastic reinforcement railing around the edges and through the center to level it with the pumice stone and sustain the weight of a consumer. The horizontal, top right side of the mat will be all pumice stone. As you shower you can soak and shuffle your feet. Under the pumice stone section will be a rubber lining with suction cups. This invention will help consumers get rid of dry, dead skin on their feet. This can be of use to people of all ages, size, and health levels.

### SEQUENCE LISTING

Not Applicable

1. The shower shuffle is a full-size shower mat with enhanced design to include a foot soaking section and a dead skin removal section. The foot soaking section will be ⅓ of the shower mat and made up of a sponge/luffa material. The remaining ⅔ of the Shower Shuffle will be made up of pumice stone for shuffling away dead skin. The bottom will have plastic railings under the sponge section to reinforce safety with weight and level it to the pumice stone. The entire bottom will contain rubber suction cups to keep the full mat in place.
